(19) United States
(12) Patent Application Publication
(10) Pub. No.: US 2024/0335133 A1
(43) Pub. Date: Oct. 10, 2024

(54) Method and Device for Monitoring Breathing Flow Based on Thoracic and Abdominal Movements

(71) Applicant: Guangzhou Medical University, Guangzhou City (CN)

(72) Inventors: Yang LIU, Guangzhou City (CN); Guoxi XIE, Guangzhou City (CN); Guijiao ZENG, Guangzhou City (CN); Ruoxuan LI, Guangzhou City (CN); Yanting LIN, Guangzhou City (CN)

(21) Appl. No.: 18/379,850

(22) Filed: Oct. 13, 2023

(30) Foreign Application Priority Data
Apr. 4, 2023 (CN) 2023103483667

(51) Int. Cl. A61B 5/08; A61B 5/00

(52) U.S. Cl. CPC A61B 5/0806; A61B 5/6805; A61B 5/7278; A61B 5/742; A61B 2562/0247; A61B 2562/046

(57) ABSTRACT

The present disclosure provides a method and device for monitoring a breathing flow based on thoracic and abdominal movements. The method includes: acquiring pressure values of pressure monitoring points of a patient, where the pressure monitoring points include a chest pressure monitoring point and an abdomen pressure monitoring point; performing data analysis based on the pressure values to determine displacement variations of the pressure monitoring points relative to initial spatial coordinates; performing nonlinear fitting based on the displacement variations to determine a thoracic volume variation and an abdominal volume variation; and determining breathing parameters based on the thoracic volume variation and the abdominal volume variation, where the breathing parameters include a total pulmonary ventilation volume, a thoracic breathing contribution ratio, an abdominal breathing contribution ratio, and a thoracic and abdominal phase difference. The present disclosure allows for breathing flow monitoring with a low cost and high accuracy.

### CROSS REFERENCE TO RELATED APPLICATION

This patent application claims the benefit and priority of Chinese Patent Application No. 2023103483667, filed with the China National Intellectual Property Administration on Apr. 4, 2023, the disclosure of which is incorporated by reference herein in its entirety as part of the present application.

### TECHNICAL FIELD

The present disclosure relates to the field of breathing monitoring, and in particular, to a method and device for monitoring a breathing flow based on thoracic and abdominal movements.

### BACKGROUND

Pulmonary ventilation function examination is one of the most basic items in breathing function examination. Many pulmonary diseases are related to ventilation function disturbance, such as chronic obstructive pulmonary diseases, asthma, and pulmonary edema. The pulmonary ventilation function examination is of important clinical significance for early screening and auxiliary diagnosis of pulmonary or airway diseases, evaluation on the tolerance of the pulmonary function to a surgery and the tolerance to labor intensity, and the like.

Existing methods special for the pulmonary ventilation function examination mainly include pulmometry and respiratory inductive plethysmography. The pulmometry, which is a traditional flow examination method, is capable of excluding pulmonary diseases such as asthma and pulmonary emphysema. However, this method requires a subject to wear a breathing mask for monitoring, causing not only a restriction to activities of the subject and an increase in respiratory airway resistance of the subject but also a change in a breathing pattern of the subject. Thus, this method is not conducive to monitoring the breathing condition of the subject for a long time. The respiratory inductive plethysmography does not require a subject to wear a breathing mask and is a non-invasive breathing measurement method. However, this method is to realize the function of pulmonary ventilation detection by measuring a cross-sectional area of the chest or the abdomen based on the electromagnetic induction principle, and has the defects of large errors and power consumption, unstable circuit, high price, and the like. Therefore, there is a need to develop an instrument or method which is low in power consumption, low in cost, simple to operate, and suitable for long-time lung monitoring, and has satisfied accuracy.

### SUMMARY

An objective of the present disclosure is to provide a method and device for monitoring a breathing flow based on thoracic and abdominal movements that can realize breathing flow monitoring with a low cost and high accuracy.

To achieve the above objective, the present disclosure provides the following technical solutions.

A method for monitoring a breathing flow based on thoracic and abdominal movements includes:
acquiring pressure values of pressure monitoring points of a patient, where the pressure monitoring points include a chest pressure monitoring point and an abdomen pressure monitoring point;
performing data analysis based on the pressure values to determine displacement variations of the pressure monitoring points relative to initial spatial coordinates;
performing nonlinear fitting based on the displacement variations to determine a thoracic volume variation and an abdominal volume variation; and
determining breathing parameters based on the thoracic volume variation and the abdominal volume variation, where the breathing parameters include a total pulmonary ventilation volume, a thoracic breathing contribution ratio, an abdominal breathing contribution ratio, and a thoracic and abdominal phase difference.

Alternatively, the performing data analysis based on the pressure values to determine displacement variations of the pressure monitoring points relative to initial spatial coordinates may specifically include:
performing data analysis based on the pressure value of the chest pressure monitoring point to determine the displacement variation of the chest pressure monitoring point relative to the initial spatial coordinates; and
performing data analysis based on the pressure value of the abdomen pressure monitoring point to determine the displacement variation of the abdomen pressure monitoring point relative to the initial spatial coordinates.

Alternatively, the performing nonlinear fitting based on the displacement variations to determine a thoracic volume variation and an abdominal volume variation may specifically include:
multiplying the area of each cross section of the trunk by a unit thickness of each cross section of the trunk to obtain a volume per unit layer thickness of the trunk;
accumulating the volume per unit layer thickness of the trunk to determine a thoracic volume at a set time and an abdominal volume at a set time;
subtracting an initial thoracic volume from the thoracic volume at a set time to obtain the thoracic volume variation; and
subtracting an initial abdominal volume from the abdominal volume at a set time to obtain the abdominal volume variation.

Alternatively, the determining breathing parameters based on the thoracic volume variation and the abdominal volume variation may specifically include:
determining the total pulmonary ventilation volume, the thoracic breathing contribution ratio, and the abdominal breathing contribution ratio based on the thoracic volume variation and the abdominal volume variation;
plotting a thoracic volume variation curve and an abdominal volume variation curve based on the thoracic volume variation and the abdominal volume variation, respectively;
acquiring an offset time between a peak of the thoracic volume variation curve and a peak of the abdominal volume variation curve; and
calculating the thoracic and abdominal phase difference based on a proportion of the offset time in a signal within a breathing cycle.

Alternatively, the determining the total pulmonary ventilation volume, the thoracic breathing contribution ratio, and the abdominal breathing contribution ratio based on the thoracic volume variation and the abdominal volume variation may specifically include:
adding up the thoracic volume variation and the abdominal volume variation to obtain the total pulmonary ventilation volume;
dividing the thoracic volume variation by the total pulmonary ventilation volume to obtain the thoracic breathing contribution ratio; and
dividing the abdominal volume variation by the total pulmonary ventilation volume to obtain the abdominal breathing contribution ratio.

The present disclosure further provides a device for monitoring a breathing flow based on thoracic and abdominal movements, using the method for monitoring a breathing flow based on thoracic and abdominal movements and including: a pressure detection system and a main control chip,

where the pressure detection system includes an elastic vest, and a plurality of piezoresistive thin-film pressure sensor units that are each disposed on an inner side of the elastic vest and configured to detect pressure values of pressure monitoring points of a patient; and the main control chip is connected to the piezoresistive thin-film pressure sensor units and configured to determine breathing parameters of the patient based on the pressure values, where the breathing parameters include a total pulmonary ventilation volume, a thoracic breathing contribution ratio, an abdominal breathing contribution ratio, and a thoracic and abdominal phase difference.

Alternatively, the pressure detection system further includes a one-out-of-sixteen gating chip and a linear voltage transformation module connected to the one-out-of-sixteen gating chip; the one-out-of-sixteen gating chip is further connected to the piezoresistive thin-film pressure sensor units; and the linear voltage transformation module is further connected to the main control chip.

Alternatively, the device for monitoring a breathing flow based on thoracic and abdominal movements further includes a screen display module connected to the main control chip and configured to display the pressure values and the breathing parameters.

Alternatively, a model of the main control chip is STM32F407ZGT6.

According to specific embodiments provided in the present disclosure, the present disclosure has the following technical effects:

According to the present disclosure, pressure values of pressure monitoring points of a patient are acquired, where the pressure monitoring points include a chest pressure monitoring point and an abdomen pressure monitoring point. Data analysis is performed based on the pressure values to determine displacement variations of the pressure monitoring points relative to initial spatial coordinates. Nonlinear fitting is performed based on the displacement variations to determine a thoracic volume variation and an abdominal volume variation. Breathing parameters are determined based on the thoracic volume variation and the abdominal volume variation, where the breathing parameters include a total pulmonary ventilation volume, a thoracic breathing contribution ratio, an abdominal breathing contribution ratio, and a thoracic and abdominal phase difference. By measuring the pressure values and utilizing the idea of a mathematical infinitesimal method, the accuracy of measuring the thoracic volume variation and the abdominal volume variation is improved, thereby allowing for high-accuracy determination of the breathing parameters. Meanwhile, since principles such as electromagnetic induction are not involved in realizing the function of pulmonary ventilation in the present disclosure, the problem of unstable resonance circuit is avoided, and due to a relatively low cost, the problems of power consumption and circuit stability of the traditional respiratory inductive plethysmography are solved.

### BRIEF DESCRIPTION OF THE DRAWINGS

To describe the technical solutions in embodiments of the present disclosure or in the prior art more clearly, the accompanying drawings required for the embodiments are briefly described below. Apparently, the accompanying drawings in the following description show merely some embodiments of the present disclosure, and those of ordinary skill in the art may still derive other accompanying drawings from these accompanying drawings without creative efforts.

FIG. 1 is a flowchart of a method for monitoring a breathing flow based on thoracic and abdominal movements provided in the present disclosure;

FIG. 2 is a diagram illustrating normal thoracic and abdominal breathing curves;

FIG. 3 is a diagram illustrating phase difference analysis of thoracic and abdominal breathing curves;

FIG. 4 is a schematic diagram of a device for monitoring a breathing flow based on thoracic and abdominal movements;

FIG. 5 is a front distribution view of pressure sensors;

FIG. 6 is a rear distribution view of pressure sensors; and

FIG. 7 is a side distribution view of pressure sensors.

### LIST OF REFERENCE NUMERALS

1—pressure detection system, 2—main control chip, and 3—screen display module.

### DETAILED DESCRIPTION OF THE EMBODIMENTS

The technical solutions of the embodiments of the present disclosure are clearly and completely described below with reference to the drawings in the embodiments of the present disclosure. Apparently, the described embodiments are merely a part rather than all of the embodiments of the present disclosure. All other embodiments derived from the embodiments in the present disclosure by a person of ordinary skill in the art without creative efforts shall fall within the protection scope of the present disclosure.

An objective of the present disclosure is to provide a method and device for monitoring a breathing flow based on thoracic and abdominal movements that can realize breathing flow monitoring with a low cost and high accuracy.

Experiments of Konno and Mead have demonstrated that in terms of pulmonary ventilation, the movements of a human respiratory cavity may be approximately treated as having two degrees of freedom in a thoracic cavity and an abdominal cavity approximately and the variation of a pulmonary volume may be equivalent to the variation of a volume of air entering the lungs via the mouth and the nose. The respiratory inductive plethysmography applied clinically is developed by establishing a basic mathematical model with the experimental conclusions. This method realizes the measurement of a pulmonary ventilation volume ΔV_{AO} by measuring and calibrating the variations of a thoracic cross-sectional area ΔuV_{RC} and an abdominal cross-sectional area ΔuV_{AB}.

ΔV_{AO}=Z·ΔuV_{RC}+L·ΔUV_{AB}

where Z and L represent movement-volume correlation coefficients of the thoracic cavity and the abdominal cavity.

In current practical engineering application, an apparatus made based on the traditional respiratory inductive plethysmography measures a cross-sectional area of a portion of the thoracic cavity and a cross-sectional area of a portion of the abdominal cavity and then substitutes the cross-sectional areas into approximate mathematical models of the thoracic cavity and the abdominal cavity to approximately calculate the volume variations of the entire thoracic cavity and the entire abdominal cavity. Inspired by this method and to improve the accuracy of non-invasive measurement of a pulmonary ventilation volume, the present disclosure innovatively proposes another method for non-invasive detection of a pulmonary ventilation volume, i.e., a breathing movement pressure-volume measurement method described below. The core idea of this method is that the thoracic cavity and the abdominal cavity of a human body will make a dilating or contracting trunk movement with pulmonary aspiration or expiration. Based on a micro-displacement generated by the trunk movement, a mathematical model of a thoracic/abdominal volume, a trunk displacement, and a breathing movement pressure is established to measure a pulmonary ventilation volume and further analyze breathing parameters.

In order to make the above objective, features, and advantages of the present disclosure clearer and more comprehensible, the present disclosure will be further described in detail below in combination with the accompanying drawings and the specific embodiments.

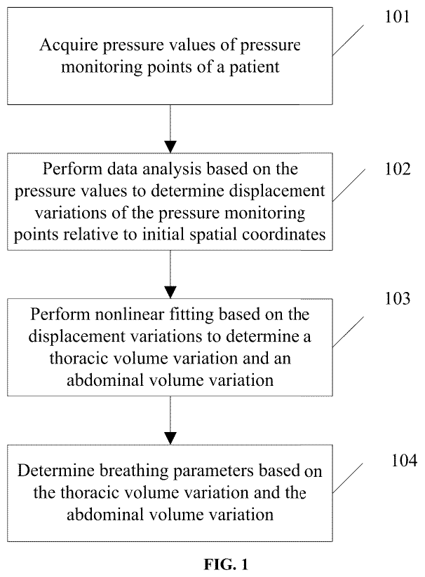

As shown in FIG. 1, the present disclosure provides a method for monitoring a breathing flow based on thoracic and abdominal movements, including:

Step 101: pressure values of pressure monitoring points of a patient are acquired, where the pressure monitoring points include a chest pressure monitoring point and an abdomen pressure monitoring point.

Step 102: data analysis is performed based on the pressure values to determine displacement variations of the pressure monitoring points relative to initial spatial coordinates.

Step 102 specifically includes: perform data analysis based on the pressure value of the chest pressure monitoring point to determine the displacement variation of the chest pressure monitoring point relative to the initial spatial coordinates; and perform data analysis based on the pressure value of the abdomen pressure monitoring point to determine the displacement variation of the abdomen pressure monitoring point relative to the initial spatial coordinates.

Step 103: nonlinear fitting is performed based on the displacement variations to determine a thoracic volume variation and an abdominal volume variation.

Step 103 specifically includes: multiply the area of each cross section of a trunk by a unit thickness of each cross section of the trunk to obtain a volume per unit layer thickness of the trunk; accumulate the volume per unit layer thickness of the trunk to determine a thoracic volume at a set time and an abdominal volume at a set time; subtract an initial thoracic volume from the thoracic volume at a set time to obtain the thoracic volume variation; and subtract an initial abdominal volume from the abdominal volume at a set time to obtain the abdominal volume variation.

Step 104: breathing parameters are determined based on the thoracic volume variation and the abdominal volume variation, where the breathing parameters include a total pulmonary ventilation volume, a thoracic breathing contribution ratio, an abdominal breathing contribution ratio, and a thoracic and abdominal phase difference.

Step 104 specifically includes: determine the total pulmonary ventilation volume, the thoracic breathing contribution ratio, and the abdominal breathing contribution ratio based on the thoracic volume variation and the abdominal volume variation; plot a thoracic volume variation curve and an abdominal volume variation curve based on the thoracic volume variation and the abdominal volume variation, respectively; acquire an offset time between a peak of the thoracic volume variation curve and a peak of the abdominal volume variation curve; and calculate the thoracic and abdominal phase difference based on a proportion of the offset time in a signal within a breathing cycle.

The determining the total pulmonary ventilation volume, the thoracic breathing contribution ratio, and the abdominal breathing contribution ratio based on the thoracic volume variation and the abdominal volume variation specifically includes:

add up the thoracic volume variation and the abdominal volume variation to obtain the total pulmonary ventilation volume; dividing the thoracic volume variation by the total pulmonary ventilation volume to obtain the thoracic breathing contribution ratio; and dividing the abdominal volume variation by the total pulmonary ventilation volume to obtain the abdominal breathing contribution ratio.

The present disclosure further provides specific operating steps of the method for monitoring a breathing flow based on thoracic and abdominal movements in practical use.

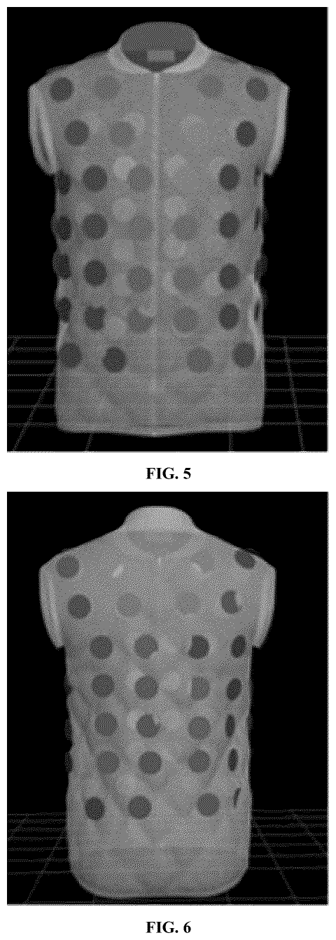

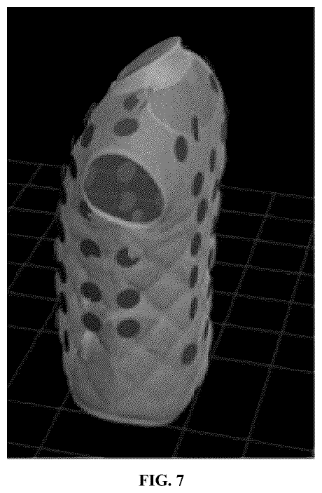

As shown in FIG. 5 to FIG. 7, a plurality of pressure sensors are integrated on an elastic vest in an array distribution manner to construct a three-dimensional pressure monitoring system for monitoring trunk movement pressures in real time. The plurality of pressure sensors should be distributed according to the following requirement: heights of spatial coordinates of pressure monitoring points at each cross section are kept identical.

A mathematical relationship between a pressure value F generated by a trunk movement and a pulmonary ventilation volume ΔV_{AO} is established as follows.

1. The elastic vest is put on a customized dynamic breathing movement dummy. Three-dimensional spatial coordinates of each pressure monitoring point at this time are defined as initial spatial coordinates of each point, and an initial thoracic volume V_{RC0} and an initial abdominal volume V_{AB0} formed by the initial spatial coordinates of each point are calculated, and the pressure value of each pressure monitoring point at this time is recorded and calibrated to “zero” (which is not actual zero because the detected pressure value is only positive).

2. In the case of controlling an air pressure to be increased or reduced by a computer program, the lungs of the dummy will be filled with or empty air to simulate the functions of the human lungs, causing a displacement at each point of the chest and the abdomen of the dummy (notes: a thoracic or abdominal movement of more than 1 cm of this dummy is programmable). The movement displacement of each monitoring point of the chest and the abdomen is recorded. The pressure value of each point of the elastic vest detected in real time is recorded. Data analysis is performed, and a mathematical relationship between a thoracic/abdominal movement displacement and a breathing movement pressure of each pressure monitoring point is established.

ΔF_{RCij}=A·Δx_{RCij}²+B·Δx_{RCij}

ΔF_{ABij}=C·Δx_{ABij}²+D·Δx_{ABij}

A and B, and C and D are thoracic and abdominal movement-volume correlation coefficients, respectively, which are related to external factors such as a vest elasticity and a dummy process and are obtained through actual calibration; ΔF_{RCij} represents a pressure variation of a monitoring point ij of the chest; ΔF_{ABij} represents a pressure variation of a monitoring point ij of the abdomen; Δx_{RCij} represents a movement displacement variation of a monitoring point ij of the chest; and Δx_{ABij} represents a movement displacement variation of a monitoring point ij of the abdomen, where i and j represent a serial number of the pressure monitoring point; if i is the same, it indicates being located at a same cross section, i.e., a spatial height in a rectangular coordinate system is the same; and j has no actual physical meaning and is only used as the serial number.

3. When controlling the dynamic breathing movement dummy to simulate the breathing movements by the computer program, an air input and an air output generated by the dummy under control are recorded simultaneously. Data analysis is performed to establish a relationship between a thoracic/abdominal displacement and a pulmonary ventilation volume: firstly, nonlinear fitting (in particular, ellipse fitting for the thoracic cavity, and circle fitting for the abdominal cavity) is performed according to two-dimensional discrete spatial coordinate points formed by the spatial movement displacements of the monitoring points at a same cross section (the displacement variation is reflected by the pressure variation, as shown in step 2) to obtain the area of each cross section of the trunk; the area of each cross section of the trunk is multiplied by a unit thickness of each cross section of the trunk to obtain a volume per unit layer thickness of the trunk; and the volume per unit layer thickness of the trunk is then accumulated to obtain a thoracic volume and an abdominal volume at a time, which are subtracted by the initial thoracic volume V_{RC0} and the initial abdominal volume V_{AB0}, respectively.

$$Δ {V}_{RC} = {\sum }_{n - 1}^{N 1} π \cdot {a}_{i} \cdot {b}_{i} \cdot Δ h - {V}_{RC 0} \\ Δ {V}_{AB} = {\sum }_{n = 1}^{N 2} π \cdot {r}_{i}^{2} \cdot Δ h - {V}_{AB 0}$$

ΔV_{RC} represents the thoracic volume variation; aᵢ and bᵢ represent a major semi-axis and a semi-minor axis of the ellipse fitted for the ith cross section, respectively; Δh represents a layer height; ΔV_{AB} represents the abdominal volume variation; rᵢ represents a radius of the circle fitted for the ith cross section, where values of aᵢ, bᵢ, and rᵢ are obtained by fitting two-dimensional spatial discrete coordinate points of a layer i; where V_{RC0} represents the initial thoracic volume, and V_{AB0} represents the initial abdominal volume; and where N1 and N2 represent numbers of distribution layers of the pressure monitoring points of the chest and the abdomen (i.e., numbers of cross section slices), respectively. The elastic vest is put on a patient. The pressure values of the thoracic and abdominal movements fed back by the elastic vest are substituted into the expression of the mathematical relationship of step 2 to obtain the displacement variation of each pressure monitoring point relative to the initial spatial coordinates. The obtained discrete point coordinates are then nonlinearly fitted by the approach in step 3 and substituted into the expression of the mathematical relationship of step 3 to obtain the thoracic and abdominal volume variations ΔV_{RC} and ΔV_{AB}. Thus, the pulmonary ventilation volume ΔV_{AO} can be obtained.

$$Δ {x}_{RCij} = \frac{- B \pm \sqrt{{B}^{2} + 4 \cdot A \cdot Δ {F}_{RCij}}}{A \cdot A} \\ Δ {x}_{ABij} = \frac{- D \pm \sqrt{{D}^{2} + 4 \cdot C \cdot Δ {F}_{ABij}}}{2 \cdot C}$$

ΔV_{AO}=ΔV_{RC}+ΔV_{AB}

The thoracic and abdominal volume variation curves can be plotted according to the obtained thoracic and abdominal volume variations, respectively, and the curves are further analyzed to acquire the breathing parameters.

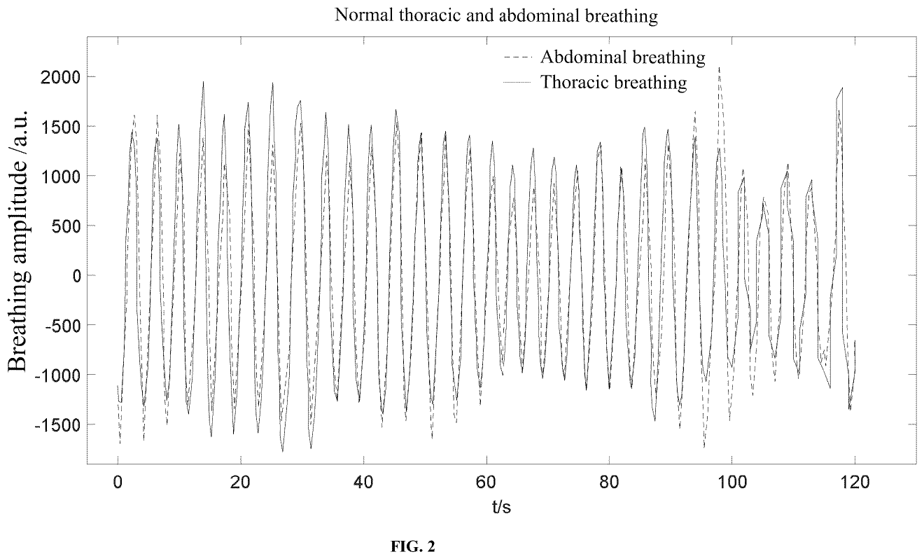

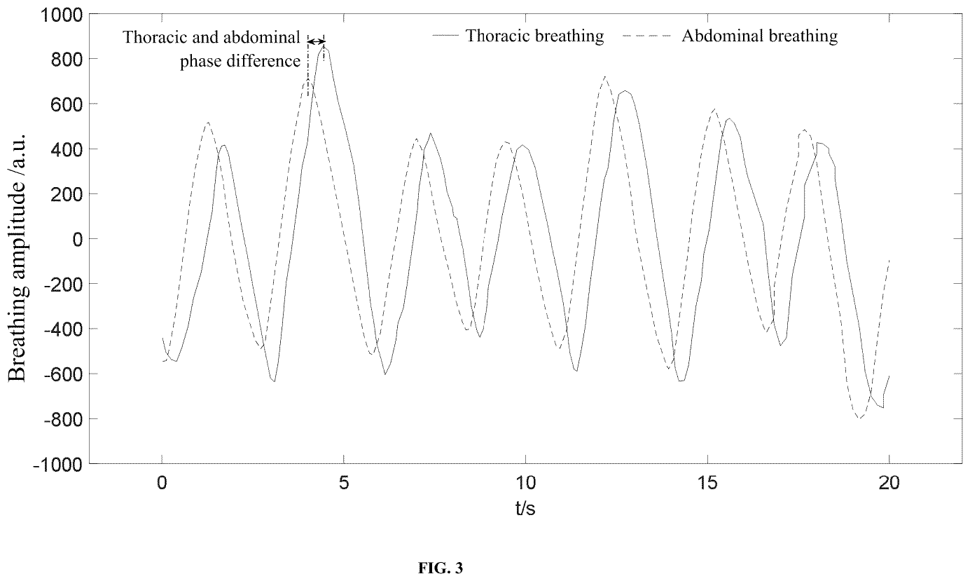

The volume variation curves are plotted according to the monitored thoracic and abdominal volume variations acquired in real time, as shown in FIG. 2 and FIG. 3, and the thoracic and abdominal breathing contribution ratios are further determined according to the thoracic and abdominal volume variations.

1. The total pulmonary ventilation volume is a sum of the thoracic volume variation and the abdominal volume variation.

2. The thoracic breathing contribution ratio is a percentage of the thoracic volume variation in the total pulmonary ventilation volume.

3. The abdominal breathing contribution ratio is a percentage of the abdominal volume variation in the total pulmonary ventilation volume.

The coordination of breathing is determined according to a phase difference between the thoracic and abdominal volume variation curves.

The thoracic and abdominal phase difference is an important parameter for determining the coordination of breathing. In the present disclosure, the phase difference is calculated by calculating the ratio of the offset time between the peak of the thoracic volume variation curve and the peak of the abdominal volume variation curve in the signal within the breathing cycle.

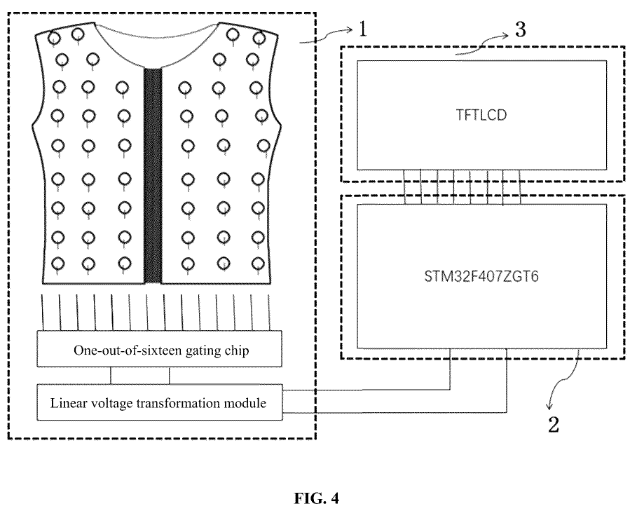

As shown in FIG. 4, the present disclosure further provides a device for monitoring a breathing flow based on thoracic and abdominal movements, using the method for monitoring a breathing flow based on thoracic and abdominal movements described above and including: a pressure detection system 1 and a main control chip 2.

The pressure detection system 1 includes an elastic vest, and a plurality of piezoresistive thin-film pressure sensor units that are each disposed on an inner side of the elastic vest and configured to detect pressure values of pressure monitoring points of a patient. The main control chip 2 is connected to the piezoresistive thin-film pressure sensor units and configured to determine breathing parameters of the patient based on the pressure values, where the breathing parameters include a total pulmonary ventilation volume, a thoracic breathing contribution ratio, an abdominal breathing contribution ratio, and a thoracic and abdominal phase difference. A model of the main control chip 2 is STM32F407ZGT6.

In practical use, the pressure detection system 1 further includes a one-out-of-sixteen gating chip and a linear voltage transformation module connected to the one-out-of-sixteen gating chip. The one-out-of-sixteen gating chip is further connected to the piezoresistive thin-film pressure sensor units. The linear voltage transformation module is further connected to the main control chip 2.

In practical use, the device for monitoring a breathing flow based on thoracic and abdominal movements further includes a screen display module 3 connected to the main control chip 2 and configured to display the pressure values and the breathing parameters.

The plurality of piezoresistive thin-film pressure sensor units are distributed in an array and fixed to the inner side of the elastic vest. Output terminals of the pressure sensors are connected to gating signal input terminals of a plurality of one-out-of-sixteen gating chips, respectively, and a common signal output terminal of the gating chips is connected to an input terminal of the linear voltage transformation module. The piezoresistive thin-film pressure sensors are configured to transform pressure signals into resistance signals. The one-out-of-sixteen gating chip is configured to sequentially select to switch on afferent signal lines of the pressure sensors, i.e., serves as a selective turn-on switch between the pressure sensors and the linear voltage transformation module. The linear voltage transformation module is configured to transform the resistance signals input after the gating chip is switched on into analog voltage signals and reasonably amplify the analog voltage signals.

An output terminal of the linear voltage transformation module is connected to an analog-to-digital converter (ADC) input pin of the board of the main control chip 2. The main control chip 2 is STM32F407ZGT6. An external ADC is disposed on the board of the main control chip 2 to transform the analog voltage signals input by the linear voltage transformation module into digital voltage signals. The main control chip 2 operates to transform the digital voltage signals into corresponding pressure data through mathematical operation. Based on the breathing movement pressure-volume measurement method described above, the mathematical model of a thoracic/abdominal volume, a trunk displacement, and a breathing movement pressure is established. It can be seen that the pressure value of each monitoring point can reflect the movement displacement of each point of the chest and the abdomen. New discrete points are formed in the rectangular space coordinate system after the displacement of each point changes. Nonlinear fitting (in particular, ellipse fitting for the thoracic cavity, and circle fitting for the abdominal cavity) is performed on the discrete points to obtain an area of each cross section of a trunk which is multiplied by a layer thickness of each cross section of the trunk to approximately obtain a thoracic volume of a layer and an abdominal volume of the layer; the thoracic volumes of layers and abdominal volumes of the layers are accumulated to obtain a total thoracic volume and a total abdominal volume; and the total thoracic volume and the total abdominal volume are subtracted by an initial thoracic volume and an initial abdominal volume to obtain the thoracic volume variation and the abdominal volume variation, thereby obtaining the pulmonary ventilation volume.

$$Δ {x}_{RCij} = \frac{- B \pm \sqrt{{B}^{2} + 4 \cdot A \cdot Δ {F}_{RCij}}}{A \cdot A} \\ Δ {x}_{ABij} = \frac{- D \pm \sqrt{{D}^{2} + 4 \cdot C \cdot Δ {F}_{ABij}}}{2 \cdot C}$$

The main control chip operates to perform nonlinear fitting to obtain values of aᵢ, bᵢ, and rᵢ of a layer i.

$$Δ {V}_{RC} = {\sum }_{n - 1}^{N 1} π \cdot {a}_{i} \cdot {b}_{i} \cdot Δ h - {V}_{RC 0} \\ Δ {V}_{AB} = {\sum }_{n = 1}^{N 2} π \cdot {r}_{i}^{2} \cdot Δ h - {V}_{AB 0}$$

ΔV_{AO}=ΔV_{RC}+ΔV_{AB}

A and B, and C and D are thoracic and abdominal movement-volume correlation coefficients, respectively, which are related to external factors such as a vest elasticity and a dummy process and are obtained through actual calibration; ΔF_{RCij} represents a pressure variation of a monitoring point ij of the chest; ΔF_{ABij} represents a pressure variation of a monitoring point ij of the abdomen; Δx_{RCij} represents a movement displacement variation of a monitoring point ij of the chest; and Δx_{ABij} represents a movement displacement variation of a monitoring point ij of the abdomen. ΔV_{RC} represents the thoracic volume variation; aᵢ and bᵢ represent a major semi-axis and a semi-minor axis of the ellipse fitted for the ith cross section, respectively; Δh represents a layer height; ΔV_{AB} represents the abdominal volume variation; rᵢ represents a radius of the circle fitted for the ith cross section, where values of aᵢ, bᵢ, and rᵢ are obtained by fitting two-dimensional spatial discrete coordinate points of a layer i; where V_{RC0} represents the initial thoracic volume, and V_{AB0} represents the initial abdominal volume; and where N1 and N2 represent numbers of distribution layers of the pressure monitoring points of the chest and the abdomen (i.e., numbers of cross section slices), respectively. ΔV_{AO} represents the pulmonary ventilation volume.

The screen display module 3 is a thin film transistor liquid crystal display (TFTLCD) screen which is directly inserted into an interface of the board of the main control chip 2. This module is configured to dynamically display the three-dimensional pressure value of each monitoring point generated by the breathing movements of the trunk in real time and display the thoracic and abdominal volume variation curves and the real-time value of the pulmonary ventilation volume at the bottom of the screen.

The operating process is described as follows.

1. The device is turned on, and a subject wears the vest.

2. The subject adjusts a posture and presses the start button on the touch screen for real-time monitoring.

3. The pressure variations generated by the real-time breathing movements of the trunk can be observed on the screen; the thoracic and abdominal volume variation curves can be observed on the screen; and breathing feature parameters such as the pulmonary ventilation volume can be read on the screen.

4. The stop button on the touch screen is pressed to return to the menu interface.

Unlike the implementation principle of an existing pulmonary ventilation volume measuring product on the market, the present disclosure uses the innovatively proposed breathing movement pressure-volume measurement method. An apparatus made based on this method supports quantitative detection of the pressure value of each region of the chest and the abdomen generated with the breathing movement and is capable of accurately measuring the breathing volume variations of the chest and the abdomen by utilizing the idea of the mathematical infinitesimal method, thus calculating the pulmonary ventilation volume.

In particular, to make the theoretically calculated pulmonary ventilation volume more accurate, unlike the traditional respiratory inductive plethysmography in which only the area of one cross section is calculated and an approximate three-dimensional mathematical model is used equivalently to the outlines of the chest and the abdomen to obtain the volume variations, in the present disclosure, the planar area of each cross section formed by the pressure monitoring points is calculated under the rectangular coordinate system and multiplied by the unit thickness of each cross section of the trunk to obtain the volume per unit layer thickness of the trunk; and the volume per unit layer thickness of the trunk is then accumulated to obtain the thoracic volume and the abdominal volume at a time, which are subtracted by the initial thoracic volume and the initial abdominal volume, respectively, thereby approximately obtaining the volume variations of the thoracic and abdominal breathing movements. Compared with the traditional respiratory inductive plethysmography, the measurement accuracy of the thoracic and abdominal volume variations can be significantly improved. Meanwhile, since principles such as electromagnetic induction are not involved in realizing the function of pulmonary ventilation in the present disclosure, the problem of unstable resonance circuit is avoided, and due to a relatively low cost, the problems of power consumption and circuit stability of the traditional respiratory inductive plethysmography are solved. Besides, the present disclosure allows for real-time three-dimensional monitoring of the breathing movement pressures of the trunk, supports real-time three-dimensional display of the pressures generated by the thoracic and abdominal breathing movements of the patient, and enhances the degree of visualization of the pulmonary ventilation function examination process.

The present disclosure utilizes a plurality of pressure sensor units that are integrated on an elastic vest in a distribution manner to construct a three-dimensional real-time monitoring system for trunk breathing movement pressures and further innovatively proposes the breathing movement pressure-volume measurement method for realize indirect real-time monitoring of the thoracic and abdominal volumes to achieve the purpose of the pulmonary ventilation volume.

The embodiments are described herein in a progressive manner. Each embodiment focuses on the difference from another embodiment, and the same and similar parts between the embodiments may refer to each other.

Specific examples are used herein for illustration of the principles and embodiments of the present disclosure. The description of the foregoing embodiments is used to help illustrate the method of the present disclosure and the core principles thereof. In addition, those of ordinary skill in the art can make various modifications in terms of specific embodiments and scope of application in accordance with the teachings of the present disclosure. In conclusion, the contents of the present description shall not be construed as limitations to the present disclosure.

What is claimed is:

1. A method for monitoring a breathing flow based on thoracic and abdominal movements, comprising:
acquiring pressure values of pressure monitoring points of a patient, wherein the pressure monitoring points comprise a chest pressure monitoring point and an abdomen pressure monitoring point;
performing data analysis based on the pressure values to determine displacement variations of the pressure monitoring points relative to initial spatial coordinates;
performing nonlinear fitting based on the displacement variations to determine a thoracic volume variation and an abdominal volume variation; and
determining breathing parameters based on the thoracic volume variation and the abdominal volume variation, wherein the breathing parameters comprise a total pulmonary ventilation volume, a thoracic breathing contribution ratio, an abdominal breathing contribution ratio, and a thoracic and abdominal phase difference.

2. The method for monitoring a breathing flow based on thoracic and abdominal movements according to claim 1, wherein the performing data analysis based on the pressure values to determine displacement variations of the pressure monitoring points relative to initial spatial coordinates specifically comprises:
performing data analysis based on the pressure value of the chest pressure monitoring point to determine the displacement variation of the chest pressure monitoring point relative to the initial spatial coordinates; and
performing data analysis based on the pressure value of the abdomen pressure monitoring point to determine the displacement variation of the abdomen pressure monitoring point relative to the initial spatial coordinates.

3. The method for monitoring a breathing flow based on thoracic and abdominal movements according to claim 1, wherein the performing nonlinear fitting based on the displacement variations to determine a thoracic volume variation and an abdominal volume variation specifically comprises:
performing nonlinear fitting based on the displacement variations to obtain an area of each cross section of a trunk;
multiplying the area of each cross section of the trunk by a unit thickness of each cross section of the trunk to obtain a volume per unit layer thickness of the trunk;
accumulating the volume per unit layer thickness of the trunk to determine a thoracic volume at a set time and an abdominal volume at a set time;
subtracting an initial thoracic volume from the thoracic volume at a set time to obtain the thoracic volume variation; and
subtracting an initial abdominal volume from the abdominal volume at a set time to obtain the abdominal volume variation.

4. The method for monitoring a breathing flow based on thoracic and abdominal movements according to claim 1, wherein the determining breathing parameters based on the thoracic volume variation and the abdominal volume variation specifically comprises:
determining the total pulmonary ventilation volume, the thoracic breathing contribution ratio, and the abdominal breathing contribution ratio based on the thoracic volume variation and the abdominal volume variation;
plotting a thoracic volume variation curve and an abdominal volume variation curve based on the thoracic volume variation and the abdominal volume variation, respectively;
acquiring an offset time between a peak of the thoracic volume variation curve and a peak of the abdominal volume variation curve; and
calculating the thoracic and abdominal phase difference based on a proportion of the offset time in a signal within a breathing cycle.

5. The method for monitoring a breathing flow based on thoracic and abdominal movements according to claim 4, wherein the determining the total pulmonary ventilation volume, the thoracic breathing contribution ratio, and the abdominal breathing contribution ratio based on the thoracic volume variation and the abdominal volume variation specifically comprises:
adding up the thoracic volume variation and the abdominal volume variation to obtain the total pulmonary ventilation volume;
dividing the thoracic volume variation by the total pulmonary ventilation volume to obtain the thoracic breathing contribution ratio; and
dividing the abdominal volume variation by the total pulmonary ventilation volume to obtain the abdominal breathing contribution ratio.

6. A device for monitoring a breathing flow based on thoracic and abdominal movements, using the method for monitoring a breathing flow based on thoracic and abdominal movements according to claim 1 and comprising: a pressure detection system and a main control chip,
wherein the pressure detection system comprises an elastic vest, and a plurality of piezoresistive thin-film pressure sensor units that are each disposed on an inner side of the elastic vest and configured to detect pressure values of pressure monitoring points of a patient; and the main control chip is connected to the piezoresistive thin-film pressure sensor units and configured to determine breathing parameters of the patient based on the pressure values, wherein the breathing parameters comprise a total pulmonary ventilation volume, a thoracic breathing contribution ratio, an abdominal breathing contribution ratio, and a thoracic and abdominal phase difference.

7. The device for monitoring a breathing flow based on thoracic and abdominal movements according to claim 6, wherein the performing data analysis based on the pressure values to determine displacement variations of the pressure monitoring points relative to initial spatial coordinates specifically comprises:
performing data analysis based on the pressure value of the chest pressure monitoring point to determine the displacement variation of the chest pressure monitoring point relative to the initial spatial coordinates; and
performing data analysis based on the pressure value of the abdomen pressure monitoring point to determine the displacement variation of the abdomen pressure monitoring point relative to the initial spatial coordinates.

8. The device for monitoring a breathing flow based on thoracic and abdominal movements according to claim 6, wherein the performing nonlinear fitting based on the displacement variations to determine a thoracic volume variation and an abdominal volume variation specifically comprises:
performing nonlinear fitting based on the displacement variations to obtain an area of each cross section of a trunk;
multiplying the area of each cross section of the trunk by a unit thickness of each cross section of the trunk to obtain a volume per unit layer thickness of the trunk;
accumulating the volume per unit layer thickness of the trunk to determine a thoracic volume at a set time and an abdominal volume at a set time;
subtracting an initial thoracic volume from the thoracic volume at a set time to obtain the thoracic volume variation; and
subtracting an initial abdominal volume from the abdominal volume at a set time to obtain the abdominal volume variation.

9. The device for monitoring a breathing flow based on thoracic and abdominal movements according to claim 6, wherein the determining breathing parameters based on the thoracic volume variation and the abdominal volume variation specifically comprises:
determining the total pulmonary ventilation volume, the thoracic breathing contribution ratio, and the abdominal breathing contribution ratio based on the thoracic volume variation and the abdominal volume variation;
plotting a thoracic volume variation curve and an abdominal volume variation curve based on the thoracic volume variation and the abdominal volume variation, respectively;
acquiring an offset time between a peak of the thoracic volume variation curve and a peak of the abdominal volume variation curve; and
calculating the thoracic and abdominal phase difference based on a proportion of the offset time in a signal within a breathing cycle.

10. The device for monitoring a breathing flow based on thoracic and abdominal movements according to claim 9, wherein the determining the total pulmonary ventilation volume, the thoracic breathing contribution ratio, and the abdominal breathing contribution ratio based on the thoracic volume variation and the abdominal volume variation specifically comprises:
adding up the thoracic volume variation and the abdominal volume variation to obtain the total pulmonary ventilation volume;
dividing the thoracic volume variation by the total pulmonary ventilation volume to obtain the thoracic breathing contribution ratio; and
dividing the abdominal volume variation by the total pulmonary ventilation volume to obtain the abdominal breathing contribution ratio.

11. The device for monitoring a breathing flow based on thoracic and abdominal movements according to claim 6, wherein the pressure detection system further comprises a one-out-of-sixteen gating chip and a linear voltage transformation module connected to the one-out-of-sixteen gating chip; the one-out-of-sixteen gating chip is further connected to the piezoresistive thin-film pressure sensor units; and the linear voltage transformation module is further connected to the main control chip.

12. The device for monitoring a breathing flow based on thoracic and abdominal movements according to claim 7, wherein the pressure detection system further comprises a one-out-of-sixteen gating chip and a linear voltage transformation module connected to the one-out-of-sixteen gating chip; the one-out-of-sixteen gating chip is further connected to the piezoresistive thin-film pressure sensor units; and the linear voltage transformation module is further connected to the main control chip.

13. The device for monitoring a breathing flow based on thoracic and abdominal movements according to claim 8, wherein the pressure detection system further comprises a one-out-of-sixteen gating chip and a linear voltage transformation module connected to the one-out-of-sixteen gating chip; the one-out-of-sixteen gating chip is further connected to the piezoresistive thin-film pressure sensor units; and the linear voltage transformation module is further connected to the main control chip.

14. The device for monitoring a breathing flow based on thoracic and abdominal movements according to claim 9, wherein the pressure detection system further comprises a one-out-of-sixteen gating chip and a linear voltage transformation module connected to the one-out-of-sixteen gating chip; the one-out-of-sixteen gating chip is further connected to the piezoresistive thin-film pressure sensor units; and the linear voltage transformation module is further connected to the main control chip.

15. The device for monitoring a breathing flow based on thoracic and abdominal movements according to claim 10, wherein the pressure detection system further comprises a one-out-of-sixteen gating chip and a linear voltage transformation module connected to the one-out-of-sixteen gating chip; the one-out-of-sixteen gating chip is further connected to the piezoresistive thin-film pressure sensor units; and the linear voltage transformation module is further connected to the main control chip.

16. The device for monitoring a breathing flow based on thoracic and abdominal movements according to claim 6, further comprising a screen display module connected to the main control chip and configured to display the pressure values and the breathing parameters.

17. The device for monitoring a breathing flow based on thoracic and abdominal movements according to claim 7, further comprising a screen display module connected to the main control chip and configured to display the pressure values and the breathing parameters.

18. The device for monitoring a breathing flow based on thoracic and abdominal movements according to claim 8, further comprising a screen display module connected to the main control chip and configured to display the pressure values and the breathing parameters.

19. The device for monitoring a breathing flow based on thoracic and abdominal movements according to claim 9, further comprising a screen display module connected to the main control chip and configured to display the pressure values and the breathing parameters.

20. The device for monitoring a breathing flow based on thoracic and abdominal movements according to claim 6, wherein a model of the main control chip is STM32F407ZGT6.
